(19) United States
(12) Patent Application Publication
(10) Pub. No.: US 2013/0023740 A1
(43) Pub. Date: Jan. 24, 2013

(54) DEVICE AND METHOD FOR MONITORING PHYSIOLOGICAL SIGNALS

(71) Applicants: Jens Kirchner, Erlangen (DE); Olaf Skerl, Bad Doberan (DE)

(72) Inventors: Jens Kirchner, Erlangen (DE); Olaf Skerl, Bad Doberan (DE)

(21) Appl. No.: 13/532,959

(22) Filed: Jun. 26, 2012

Related U.S. Application Data
(60) Provisional application No. 61/510,084, filed on Jul. 21, 2011.

(51) Int. Cl. A61B 5/0205; A61B 5/11; A61B 5/00

(52) U.S. Cl. 600/301

(57) ABSTRACT

An implantable device includes a posture sensor and a physiological signal sensor. The posture sensor supplies at least one first sensor output signal indicating body posture and/or changes therein. The physiological signal sensor supplies at least one second sensor output signal indicative of at least one physiological parameter such as blood pressure, intracardiac impedance, stroke volume, heart sounds, heart rate, and/or biochemical measurements (e.g., oxygen concentration). An evaluation unit processes the first and second sensor output signals and determines one or more variables that describe the dynamic behavior of the second sensor output signal in response to a change in body posture indicated by the first sensor output signal.

### CROSS-REFERENCE TO RELATED APPLICATIONS

This application claims priority under 35 USC §119(e) to U.S. Provisional Patent Application 61/510,084 filed Jul. 21, 2011, the entirety of which is incorporated by reference herein.

### FIELD OF THE INVENTION

The invention generally relates to devices for measuring heart health, and more particularly to implantable devices which use physiological signal sensors and posture sensors to measure the body's behavior upon experiencing a posture change.

### BACKGROUND OF THE INVENTION

Prior devices which detect the response of a human body to a change in posture tend to use one of the following approaches:
1. Measuring and analyzing the intracardiac and intrathoracic impedance
2. For blood pressure measurement: systolic, diastolic and mean blood pressure
3. Combining blood pressure measurement and postural change: difference in the mean pressures between two body postures

The solutions mentioned above in 1 and 2 are primarily directed to measurement and interpretation of changes in the global and static behavior of the measured variable, i.e., such changes are detected if they persist in all stress situations.

As known from US 2007/0156057, the static values of the detected physiological parameters for various body postures are those which develop as a result of the respective body postures—they are “steady state values” for the postures, so to speak.

When using blood pressure signals, often times the use of the relative pressure is required (the measured absolute pressure minus ambient pressure). This requires an additional sensor system for detecting the ambient pressure, which can entail further disadvantages.

### SUMMARY OF THE INVENTION

The invention is intended to provide improved detection of heart diseases such as CHF (congestive heart failure). One aspect of the invention involves an implantable device including a posture sensor and a physiological signal sensor. In the implanted state, the posture sensor supplies a first sensor output signal indicating a body posture and/or a change in a body posture. The physiological signal sensor detects a physiological parameter, the value of which changes in a healthy person in response to a change in the body posture (for example standing, sitting, lying down and the like). Such a physiological parameter can be, for example, blood pressure, intracardiac impedance, stroke volume, heart sounds, heart rate, biochemical signals and/or oxygen concentration. The physiological signal sensor supplies a second sensor output signal which reflects the physiological parameter or parameters.

The posture sensor and the physiological signal sensor are connected to an evaluation unit which is designed to process the first and second sensor output signals and determine, based on these output signals, one or more variables that describe the dynamic behavior of the second sensor output signal in response to a change in body posture indicated by the first sensor output signal. This dynamic behavior is of interest because it reflects heart health: changes in body posture lead to redistribution of the blood volume in the body due to gravity, and the redistribution is offset by short-term compensation mechanisms of the cardiovascular system. Thus, the response behavior of the compensation mechanisms reflect the cardiovascular system's performance, and in particular the state of health of the heart.

Unlike prior systems for measuring posture-dependent physiological variables, the invention involves the idea of detecting the dynamics of the change of a detected physiological parameter in response to a posture change—which is to comparable to a “step response” in the control engineering sense—instead of merely detecting steady state error of the respective detected physiological parameter in response to a postural change.

The invention includes the realization that in prior systems, changes are often not detected until a very late stage of the disease, and that the reduced performance of the cardiovascular system, particularly in an early stage of the disease, is not readily apparent. By utilizing dynamic change of a posture-dependent physiological parameter, the invention recognizes that the body must expend effort to achieve a change in the parameter in response to posture changes. The temporal course of the physiological parameter in response to a change in the body posture thus provides additional information about the performance of the cardiovascular system.

A (preferably implantable) device according to the invention preferably includes a 3-axis accelerometer, a blood pressure sensor, and an evaluation unit. It detects suitable postural changes via the acceleration sensor, determines the response in the blood pressure signal (i.e., the “step response” of the endogenous compensation mechanism), and extracts dynamic parameters of the compensation mechanism. These serve as a measure of the regulatory performance of the heart. They can be used to detect and monitor CHF and as prediction parameters. In a sense, the invention utilizes methods similar to those in controls engineering wherein systems are identified by means of their step response.

A special field of use of the device is the detection and observation of the status and progress of cardiac insufficiency, e.g., congestive heart failure (CHF)—in other words, the monitoring of CHF—wherein the dynamics in the response behavior of physiological parameters to body posture changes is observed.

A preferred version of the device includes at least one sensor and an evaluation unit, which carry out the following method:
(a) continuously measuring a variable which detects the body posture or the change thereof (first sensor output signal);
(b) detecting a change in the body posture, and optionally determining the type and/or the degree of this change, based on the first sensor output signal;
(c) continuously or intermittently measuring at least one of the following physiological signals: blood pressure, intracardiac impedance, stroke volume, heart sounds, heart rate, biochemical signals, oxygen concentration, as the second sensor output signal;
(d) preferably triggering the measurement of the second sensor output signal or selection of a suitable time segment based on the detection of the postural change from the first sensor signal; and
(e) calculating one or more measures (variables) which describe the dynamic behavior of the response of the second sensor output signal to the new body posture (for example, increases, time constants or delays), and optionally including the type/degree of the postural change and/or additional sensor variables.

The detection of the patient posture is preferably implemented by a 3D acceleration sensor. As an alternative, the patient posture, and more particularly the change thereof, can also be detected based on the second sensor output signal, so that the posture sensor and the physiological signal sensor can be implemented as a single sensor.

The evaluation unit is preferably designed to divide the response signal into sub-intervals and to further evaluate individual sub-intervals.

The evaluation unit can be designed, for example, to carry out the aforementioned method, wherein in the foregoing step (e), the time interval determined in step (d) is divided into several sub-intervals. The variables that describe the dynamics of the compensation are preferably derived in at least one of the sub-intervals.

To this end, preferably at least one of the following variables is determined:
Delays, for example, the time between the postural change and the start of compensation;
Slopes of the signal at certain times, or in certain time segments (for example, pressure drop/time); values that are averaged over the time segment or extreme values can be determined;
Duration of characteristic sub-intervals of the signal curve;
Duration that the signal requires in a given time segment to pass through a given relative signal difference (which is to say, the approximated determination of a time constant).

Preferably step (e) additionally includes the fit of a model function to at least a segment of the signal. The variables determined in step (e) are then suitable parameters of this model function. An exponential function, for example, can be used as the underlying model function for the dynamic behavior of the response of the second sensor output signal to the new body posture, for example:

$$\frac{P ( t )}{Δ P} = 1 - {e}^{\frac{t}{τ}}$$

with ΔP describing the change in the physical variable due to the postural change. The time constant τ, for example, is determined as a suitable parameter of the model function using known methods. A change in the time constant (for example, an increase) provides an early indication of a change in the performance of the compensation mechanism (for example, worsening).

When fitting a given model function, the parameters thereof are of interest, for example the time constants. In addition, the shape of the response behavior is important, i.e., whether (for example) the course of the signal of the response to the new body posture is linear, exponential, sigmoid, with one or more overshoots, oscillating and the like. For this purpose, different model functions are preferably fitted to the signal segment and the quality of these fits is compared. It is of diagnostic interest which of the models agrees best with the measured data. The fit parameters for this model can also be analyzed.

The determined parameters can be recorded as a trend or they can be compared to threshold values. Trend parameters can be derived from the trends and likewise be compared to threshold values. For this purpose, the device preferably includes a memory and is also preferably equipped with a telemetry unit which makes it possible to transmit measurement and parameter values to a central service center, for example for further evaluation by analysis by a physician. The determined measurement and parameter values can be utilized as predictors for predicting disease events or used directly by the device for therapy control and optimization. This is advantageous when the device is part of an implantable cardiac stimulator, such as a cardiac pacemaker or cardioverter/defibrillator (ICD), for example.

### BRIEF DESCRIPTION OF THE DRAWINGS

The invention will now be described in more detail based on exemplary versions depicted in the accompanying drawings, wherein:

FIG. 1 shows an implantable medical device 10, here an implantable cardiac stimulator, and an implantable electrode line 20 connected thereto.

FIG. 2 shows several exemplary components of an implantable medical device according to the invention (e.g., the device shown in FIG. 1).

FIG. 3 shows a schematic curve of the mean blood pressure P over the time t during a postural change.

FIG. 4 shows schematic curves of the mean blood pressure P over the time t during a postural change.

### DETAILED DESCRIPTION OF EXEMPLARY VERSIONS OF THE INVENTION

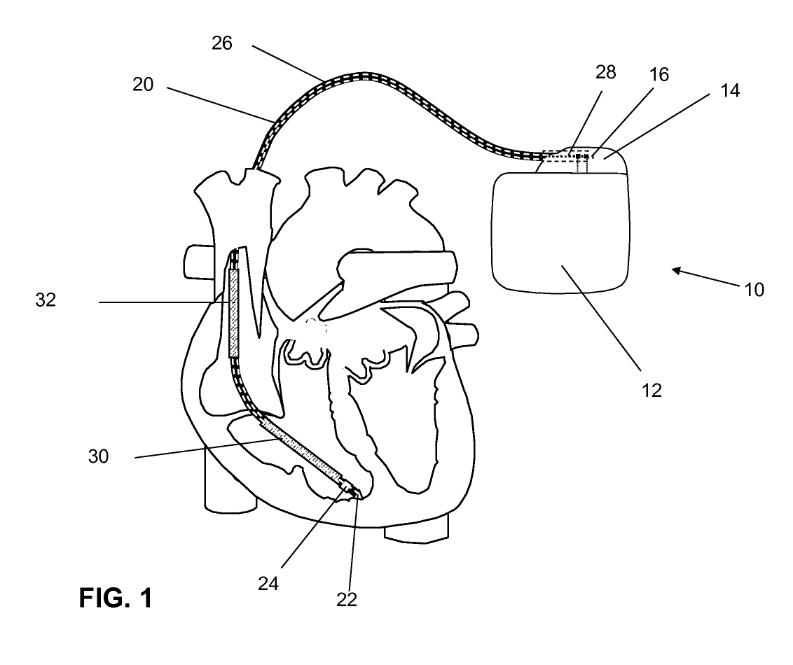

FIG. 1 shows an implantable medical device in the form of an implantable cardiac stimulator 10, to which an electrode line 20 is connected. The implantable cardiac stimulator 10 can be a cardiac pacemaker or a cardioverter/defibrillator (ICD). In the version shown in FIG. 1, the cardiac stimulator 10 is a ventricular cardiac pacemaker and defibrillator. Other known cardiac stimulators are dual-chamber cardiac pacemakers for stimulating the right atrium and right ventricle, or biventricular cardiac pacemakers, which can stimulate the left ventricle in addition to the right ventricle. It is noted that while the following discussion will focus on the invention as embodied in a right-ventricular cardiac pacemaker and defibrillator, the invention could be embodied in any other suitable electromedical implant, e.g., a multi-chamber cardiac pacemaker or cardioverter/defibrillator (ICD), a neurostimulator, or a pure monitoring implant.

Implantable cardiac stimulators of the type shown typically include a housing 12, which is generally made of metal and is therefore electrically conductive and can be used as a large-surface-area electrode pole. Typically, a terminal housing 14, which is also referred to as a header, is fastened to the outside of the housing 12. Such a header typically includes contact bushings for receiving plug contacts. The contact bushings include electric contacts 16, which are connected to an electronics unit disposed in the housing 12 of the cardiac pacemaker 10 by way of corresponding conductors.

The distal end of the electrode line 20 includes electrode poles in the form of a tip electrode 22 disposed at a distal end of the electrode line 20, and an annular electrode 24 disposed in the vicinity of the tip electrode 22. Depending on the function of the cardiac stimulator to which the electrode line 20 is connected, the electrode poles 22 and 24 are designed to be used, for sensing electric potentials of the heart tissue (myocardium) or for delivering electric signals, for example for delivering stimulation pulses to the surrounding heart tissue. FIG. 1 shows how the electrode poles, these being the tip electrode 22 and the annular electrode 24, are located in the apex of the right ventricle of a heart when the electrode line 20 is used.

Both the tip electrode 22 and the annular electrode 24 are electrically connected to a contact of a connector 28 at the proximal end of the electrode line 20 by way of at least one electric conductor 26. The connector 28 includes electric contacts which correspond to the electric contacts 16 of the contact bushing in the terminal housing 14 of the implantable cardiac stimulator.

The electric conductors 26 in the electrode line 20 can be designed as approximately elongated sheathed cable conductors or helically coiled conductors. Such conductors, which connect functional electrode poles to electric contacts of the plug contact at the proximal end of the electrode line 20 in an electrically conductive manner, are used to transmit electric signals from the plug contact to the respective electrode pole, or to conduct sensed signals representing electric potentials from the respective electrode pole to the plug contact.

The electric conductors 26, which connect the electrode poles 22 or 24 to the electric contact of the connector 28 of the electrode line 20, are surrounded by an insulating jacket over the majority of the lengths thereof, so that an electric contact with the tissue of the heart is limited to the electrode poles.

In addition to the electrode poles 22 and 24, which are typically used for (in this case ventricular) stimulation of the heart tissue, the electrode line 20 also includes two larger-surface-area electrode poles 30 and 32, which serve as defibrillation electrodes and which are formed by at least one exposed helically coiled wire.

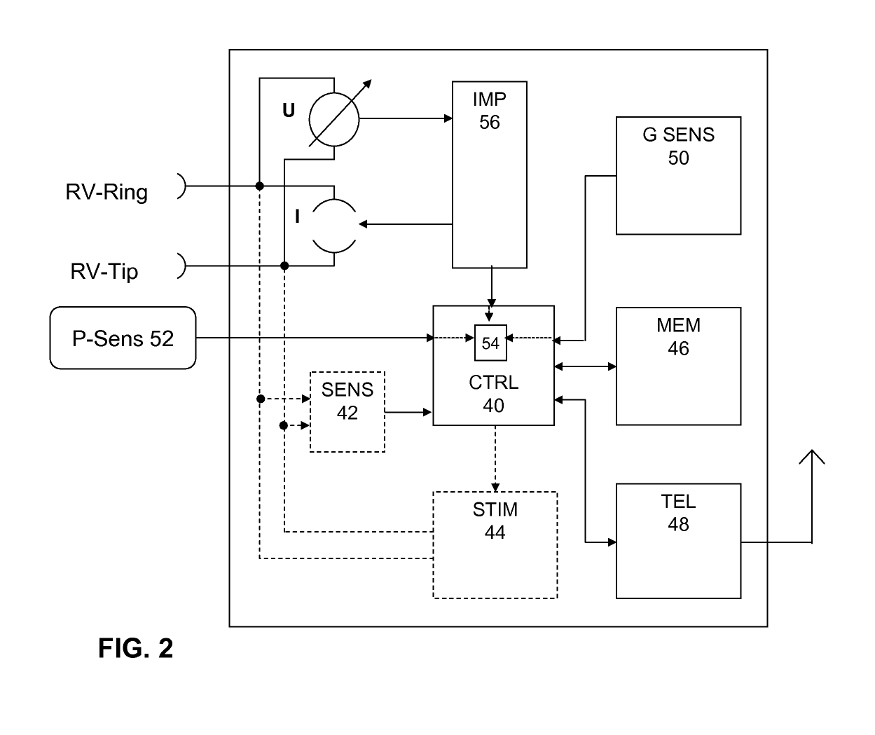

FIG. 2 is a schematic illustration of several exemplary components of the cardiac stimulator 10 from FIG. 1. Typical components of such a cardiac stimulator are a control unit 40, one or more sensing units 42 which each represent a diagnostic unit, and one or more stimulation units 44 which each represent a treatment unit. The control unit 40 is connected to both the sensing unit 42 and the stimulation unit 44. Both the sensing unit 42 and the stimulation unit 44 are connected to electrode terminals, respectively, so that the sensing unit 42 is able to capture electric potentials of the heart tissue by way of the right ventricular annular electrode 24 and/or the right ventricular tip electrode 22, and the stimulation unit 44 is able to deliver stimulation pulses by way of the right ventricular annular electrode 24 and/or the right ventricular tip electrode 22.

In addition, the control unit 40 is connected to a memory unit 46 for storing captured values of parameters to be measured. A telemetry unit 48, which is likewise connected to the control unit 40, allows captured values of parameters to be transmitted to an external device, and/or allows control commands to be received from an external device.

The control unit 40 is moreover connected to a 3D acceleration sensor (3D accelerometer) 50, which is designed to detect not only dynamic acceleration, for example during physical activity, but also a respective device position, which in the implanted state of the device corresponds to a respective body posture. The 3D acceleration sensor thus serves as a posture sensor.

In addition to the posture sensor, the electromedical implant includes at least one physiological signal sensor 52 which detects a physiological parameter, the value of which changes in response to a change in the body posture (e.g., changes between standing, lying down, sitting, or other body positions). Such a physiological parameter is, for example, the blood pressure, the intracardiac impedance, the stroke volume, cardiac sounds, the heart rate, biochemical signals and/or the oxygen concentration. In FIG. 2, the physiological signal sensor 52 is a blood pressure sensor 52, which is preferably designed to detect the blood pressure in the pulmonary artery and supply a corresponding output signal (one representing blood pressure) to the control unit 40. The blood pressure sensor 52 serves as a physiological signal sensor.

An evaluation unit 54, which is connected at least indirectly to the blood pressure sensor 52 and the 3D acceleration sensor 50 (and which can thus evaluate the output signals of these sensors, as will be described in more detail below), is part of the control unit 40.

The control unit 40 is additionally connected to an impedance determination unit 56. The impedance determination unit 56 is connected to a power source I and a voltage measuring unit U, which in turn are connected to the terminals for the annular electrode 24 and the tip electrode 22. In this way, the direct current source I can constantly deliver voltage pulses by way of the tip electrode 22 and the annular electrode 24, and the voltage measuring unit U can measure the respective voltage that is released. On the basis of these values, the impedance determination unit 56 can determine a particular impedance value. An impedance value determined in this way depends on a variety of influencing variables. For example, a fracture of an electric conductor in the electrode line 20 would manifest itself in a very high impedance value. When the electrode line 20 is intact, the impedance to be measured between the electrode poles 22 and 24 also depends on the blood volume in the right ventricle of a heart, so that the impedance to be measured cyclically fluctuates in accordance with the cardiac cycle. For example, the impedance increases as the blood volume decreases, which is to say as the volume of the right ventricle decreases, so that a cyclical rise in the impedance indicates the cyclical contraction of the right ventricle. Likewise, a corresponding rise of the measured impedance due to ventricular contraction can indicate successful stimulation after delivery of a stimulation pulse. In this way, the impedance determination unit 56 is able to carry out automatic stimulation success control (automatic capture control (ACC)).

The impedance that is measured additionally depends on the impedance of the electrode pole-tissue contact. By evaluating the measured impedance values, it is therefore also possible to detect the formation of edema, which can occur, for example, by heating of the electrode poles due to alternating magnetic fields of a nuclear magnetic resonance tomograph.

The postural sensor and physiological signal sensor can take forms different from the 3D acceleration sensor 50 and the blood pressure sensor 52, and/or may include multiple sensors, and/or may be used for multiple purposes. As one example, the impedance determination unit 56 could be used instead of (or in addition to) the blood pressure sensor 52 as the physiological signal sensor. As another example, the blood pressure sensor 52 can be used in conjunction with an activity sensor, and could be used for adaptation of the cardiac stimulation rate by means of the control unit 40.

The acceleration sensor 50 and blood pressure sensor 52 are connected to the evaluation unit 54, as is shown in FIG. 2. The acceleration sensor 50 and the blood pressure sensor 52 continuously or cyclically supply signals that reflect the acceleration vector detected by the acceleration sensor or blood pressure values. The blood pressure values are preferably values of the blood pressure in the pulmonary artery.

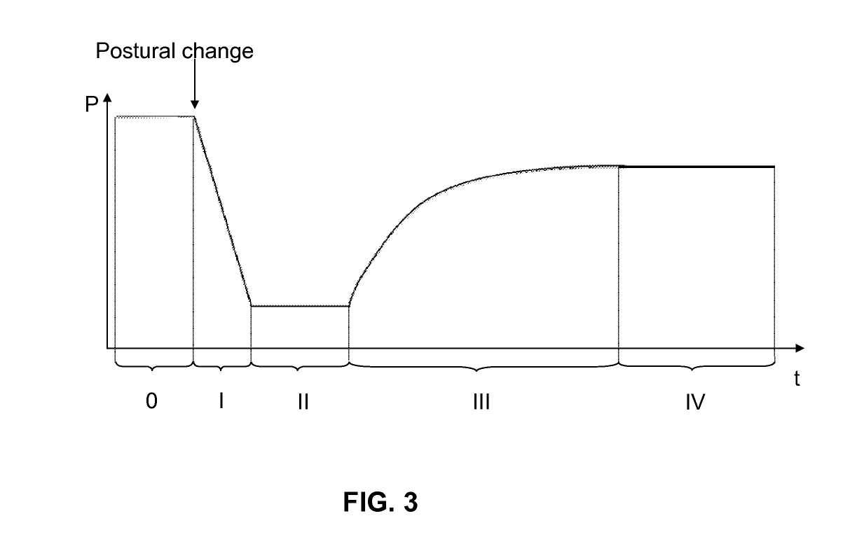

FIG. 3 schematically shows the curve of a physiological signal—for example the blood pressure—over the time t during a postural change, for example when getting up from a lying posture to a standing posture.

To this end, different phases must be distinguished, the durations of which are indicated in FIG. 3 beneath the time axis t:

### Phase 0. Equilibrium Phase Prior to the Postural Change

During this period, the physiological signal has a certain static value.

### Phase I. Change in the Physiological Signal Due to a Postural Change.

This change is caused, for example, by the gravity-related redistribution of the blood volume when getting up. The blood settles into the legs, causing the blood pressure measured in the torso to briefly drop.

### Phase II. Delay

The delay phase is the “dead time” before response, i.e., the time period between the change in the physiological signal and the start of the effect of the endogenous compensation mechanisms.

### Phase III. Compensation

During this period, the body's endogenous compensation mechanisms attempt to correct the deviation caused by the postural change, for example, to readjust thoracic blood pressure.

### Phase IV. Equilibrium Phase Following the Postural Change

After successful regulation, the new static state follows. After the correction of Phase III has been made, the value of the physiological parameter can deviate from the “steady state error” value in Phase 0, prior to the postural change.

As mentioned above, prior approaches focused on the “steady state error,” rather than on one or more of Phases I-III. In contrast, the evaluation unit 54 is designed to determine the dynamic parameters of the control process, i.e., parameters of the step response, particularly in Phase III (though features of phase II, and possibly Phase I, may be used as well). This approach has two principal advantages: disturbances in the body's compensation mechanisms become apparent in an early stage in the dynamic parameters, long before a change occurs in the static parameters (i.e., long before any differences between phases 0 and IV are apparent). And no long-term stable absolute values, or absolute values compensated for with a reference, are required for determining the dynamic parameters.

The evaluation unit preferably carries out the following in response to a detection of a postural change based on the signals of the postural sensor:
Determining the time interval to be analyzed for quantifying the compensation processes, in particular starting after Phase I's gravity-related blood redistribution until a new equilibrium state of the blood pressure is reached in Phase IV.
Calculating a suitable variable for this time interval that describes the dynamics of the compensation for the blood redistribution, for example increase in the blood pressure drop in Phase I, time duration until the new equilibrium state is reached, delay until the compensation process starts, suitable fit parameters (for example time constant of an exponential fit function), and the like.

These variables supply diagnostic information about the performance of the compensation mechanisms, which decreases, for example, as the cardiac insufficiency increases.

A variety of different approaches can be used for detection of changes in body posture. Changes in posture are preferably detected by means of a 3D acceleration sensor, but as an alternative, the postural change can be derived from a blood pressure signal or blood flow signal by detecting sudden changes (for example, by means of a threshold value for the pressure change and/or a test of a fit function). Thus, the blood pressure sensor (or blood flow sensor) can also serve as a postural sensor. As an alternative, an external sensor system can be used, such as a camera or other imaging system, or one or more pressure sensors situated on a bed, chair, and/or other surfaces which report when the patient sits down on the chair, lies down on the bed, and the like.

For the non-postural physiological parameter, while blood pressure is the preferred parameter, others such as blood flow, the heart rate or heart sounds, stroke volume, intracardiac impedance, oxygen concentration or other biochemical signals, etc. may alternatively or additionally be used. When evaluating a change in the blood pressure, it is preferable to focus analysis on the gravity-related redistribution of the blood volume (Phases II & III). However, the redistribution can be analyzed during the time interval from the postural change until the start of compensation processes by the cardiovascular system (Phase I and potentially Phase II), or from the postural change until new steady-state values are reached (Phases I-III).

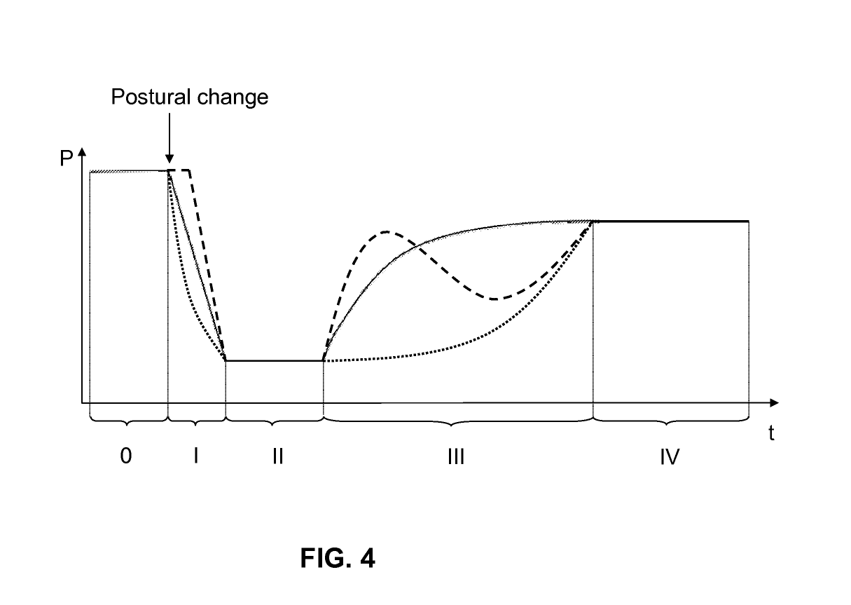

Examples of different curve progressions will now be reviewed. FIG. 4 schematically shows two additional exemplary pressure curves, which are illustrated by the dashed and dotted lines in comparison with the pressure curve shown in FIG. 3. For clarity reasons, the durations of the individual phases in this example are the same for all pressure curves shown.

Delays: The time durations of the individual phases, in particular Phases II and III, can vary. The delay prior to the start of the compensation processes and prior to the renewed rise of the signal (i.e., the Phase II “dead time”), and the duration of the compensation (Phase III), supply diagnostic information about the regulatory performance of the organism. Phase II may be absent, for example if the compensation starts immediately without delay.

Different progressions: During Phase I, the signal may decrease in an exponential curve (dotted line) rather than linearly. Additionally, as shown by the dashed line, a delay may occur in Phase I after the postural change and before the change in the physiological parameter becomes visible. Differently-shaped curves can occur in the compensation phase III (indicated by the dotted and dashed lines), such as exponential, sigmoid and the like. It is also possible to have a graduated increase over several stages.

Variable rates during drop/increase: In addition to the durations of the individual phases and the curves of the physiological signal during such phases, it is useful to analyze the ratio of the values of the physiological signal at the beginning and end of the phases. For example, the slope of the physiological signal can be determined during the drop or increase.

A preferred version of the device carries out the following method steps:
Simultaneously continuously measuring the blood pressure and acceleration;
Detecting a postural change based on the measured acceleration;
Evaluating the blood pressure curve starting with a detected postural change, for example, for an interval of 3 minutes;
Determining the minimum (or maximum) of the curve so as to determine the end of the gravitation-related blood redistribution;
Testing whether the pressure remains constant for a period of time around this minimum. This period may be stored as the delay, and can be used as a parameter that is of diagnostic interest.
Fitting various fit functions to the sub-curve following the curve minimum or at the end of the delay, for example linear, exponential, sigmoid curve and the like. The quality of these fits (for example, values derived from a x² value) can be compared, and the optimal fit function can be determined. A model function, for example an exponential function, could instead be assumed a priori. The fit function is of diagnostic interest, as are the parameters of this function (which describe the dynamics of the compensation process).

Additional benefits can arise if further physiological parameter sensors are added. Adding further sensors can increase the specificity of the evaluation of the compensation processes. In this way, for example, stress situations can be distinguished which cannot be sufficiently determined by an activity sensor. In particular, a postural change during physical activity can be expected to be compensated for more quickly than after an extended resting phase, and it is therefore useful to measure heart rate to more easily differentiate between compensation at various resting and activity ranges. Analogously, the mean blood pressure can also be used to distinguish different stress situations.

The invention is preferably used to detect and monitor cardiac insufficiency (CHF). As an alternative, the methods described can also be used to characterize the condition of other components regulating the blood pressure, e.g., defects of the venous valves. In addition, the effect of pharmaceuticals or medical devices that influence such control mechanisms, for example baroreflex stimulators, can be monitored.

In addition to using the invention for monitoring purposes, the calculated variables can also be used to adapt the behavior of medical devices, for example pacemaker functions or the dosing of (automatic) pharmaceutical dispensing.

The preferred versions of the invention discussed above focus on the response of the cardiovascular system to specific changes in stress, and thus allow changes in the performance of the heart to be detected. These changes are slow to manifest themselves in the global static behavior of the measured variable. The invention is thus more sensitive than methods that are based, for example, on 24-hour mean values of a variable.

Moreover, it is not static target values that are analyzed, which notably in the initial phase of a disease are not yet strongly influenced, but rather the dynamic parameters of the body's compensation process. Changes in compensation performance become visible in the dynamic behavior at a very early stage, so that greater sensitivity is to be expected than with other methods.

Because short segments of the physiological parameter—and in most cases only changes in the parameter—are analyzed, calibration of the parameter measurements is not required. A patient's stress level is accounted for, and measurements are not influenced by the patient's daily schedule, and periods of relative activity and inactivity. Notably, blood pressure signals—which are not based on the ambient pressure—can be used. This eliminates effects from compensation for ambient pressure changes, for example due to changes in weather or a change in the elevation position of the patient.

Exemplary versions of the invention have been described above in order to illustrate how to make and use the invention. It will be apparent to those skilled in the art that numerous modifications and variations of the described versions are possible in light of the foregoing discussion. The invention is not intended to be limited to these versions, but rather is intended to be limited only by the claims set out below. Thus, the invention encompasses all different versions that fall literally or equivalently within the scope of these claims.

What is claimed is:

1. A monitoring device including:
a. a posture sensor configured to provide a first sensor output signal indicating at least one of:
(1) body posture, and
(2) a change in body posture,
b. a physiological signal sensor configured to provide a second sensor output signal indicating a value of a physiological parameter;
c. an evaluation unit configured to determine at least one variable characterizing the dynamic behavior of the second sensor output signal in response to a change in body posture indicated by the first sensor output signal.

2. The monitoring device of claim 1 wherein the physiological parameter includes at least one of:
a. blood pressure,
b. intracardiac impedance,
c. stroke volume,
d. heart sounds,
e. heart rate, and
f. oxygen concentration.

3. The monitoring device of claim 1 wherein the posture sensor is a 3D acceleration sensor.

4. The monitoring device of claim 1 wherein the evaluation unit is designed to identify, from the first sensor output signal, a time of a change in a body posture.

5. The monitoring device of claim 1 wherein the physiological signal sensor is a blood pressure sensor.

6. The monitoring device of claim 1 wherein the physiological signal sensor is an impedance sensor.

7. The monitoring device of claim 1 wherein the physiological signal sensor is configured to initiate provision of the second sensor output signal upon or following a change in body posture indicated by the first sensor output signal.

8. The monitoring device of claim 1 wherein the evaluation unit is configured to:
a. divide the second sensor output signal into sub-intervals ordered in succession over time, and
b. further determine, for one or more of the sub-segments, at least one variable characterizing the dynamic behavior of the second sensor output signal during each sub-interval.

9. The monitoring device of claim 8 wherein the evaluation unit is further configured to determine a duration of a sub-interval of the second sensor output signal.

10. The monitoring device of claim 1 wherein the evaluation unit is configured to quantify a delay between:
a. a value of the first sensor output signal indicative of a change in body posture, and
b. the start of a change of the second output signal.

11. The monitoring device of claim 1 wherein the evaluation unit is configured to quantify a slope of the second sensor output signal following a value of the first sensor output signal indicative of a change in body posture.

12. The monitoring device of claim 1 wherein the evaluation unit is configured to determine a duration required for the second sensor output signal to pass through a given second sensor output signal difference.

13. The monitoring device of claim 1 wherein the evaluation unit is configured to:
a. fit a model function to the second sensor output signal, and
b. identify one or more function parameters characterizing the model function.

14. The monitoring device of claim 1 wherein the monitoring device is a cardiac stimulator.

15. A monitoring method including the following steps:
a. at least substantially continuously measuring a first sensor output signal indicating at least one of:
(1) body posture, and
(2) a change in body posture,
b. detecting a change in body posture indicated by the first sensor output signal;
c. measuring a second sensor output signal indicating a value of a physiological parameter;
d. calculating at least one variable characterizing the dynamic behavior of the second sensor output signal in response to a change in body posture indicated by the first sensor output signal.

16. The monitoring method of claim 15 wherein the physiological parameter includes at least one of:
a. blood pressure,
b. intracardiac impedance,
c. stroke volume,
d. heart sounds,
e. heart rate, and
f. oxygen concentration.

17. The monitoring method of claim 15 further including the step of initiating measurement of the second sensor output signal upon or following a change in body posture indicated by the first sensor output signal.

18. The monitoring method of claim 15 wherein the calculated variable characterizing the dynamic behavior of the second sensor output signal is a parameter characterizing a fit of a model function to at least a portion of the second sensor output signal.

19. The monitoring method of claim 18 wherein the model function is an exponential function.

20. A monitoring device including:
a. a posture sensor;
b. a physiological signal sensor;
c. an evaluation unit configured to:
(1) receive from the posture sensor a first sensor output signal characterizing body posture;
(2) receive from the physiological signal sensor a second sensor output signal characterizing one or more of:
(a) blood pressure,
(b) blood flow,
(c) stroke volume,
(d) heart sounds,
(e) heart rate, and
(f) oxygen concentration, and
(b) intracardiac impedance;
(3) characterize the behavior of the second sensor output signal in response to a change in body posture indicated by the first sensor output signal.
